(19) United States
(12) Patent Application Publication
(10) Pub. No.: US 2025/0076271 A1
(43) Pub. Date: Mar. 6, 2025

(54) MODULAR SENSOR INTEGRATION SYSTEM WITH SELECTABLE SENSOR MODULES

(71) Applicant: Buckman Laboratories International, Inc., Memphis, TN (US)

(72) Inventors: David Alan Ambrose, Memphis, TN (US); David John Howard, Memphis, TN (US); William Anthony Von Drasek, Memphis, TN (US)

(21) Appl. No.: 18/815,068

(22) Filed: Aug. 26, 2024

Related U.S. Application Data
(60) Provisional application No. 63/535,455, filed on Aug. 30, 2023.

(51) Int. Cl. G01N 33/18; G01D 21/02; G05B 15/02

(52) U.S. Cl. CPC G01N 33/1886; G01D 21/02; G05B 15/02

(57) ABSTRACT

A diverse sensor integration system includes a system controller, at least one system sensor, and a plurality of selectable sensor modules. Each selectable sensor module is operably connected between the system controller and the system sensor. The selectable sensor modules receive an input signal with a set of first characteristics from the system sensor and convert it into an output signal with a set of second signal characteristics required by the system controller. The output signal is then transmitted to the system controller. This integration system allows for the seamless integration of different types of sensors into a single system, providing flexibility and adaptability to meet various system requirements.

### CROSS-REFERENCES TO RELATED APPLICATIONS

This application claims benefit of U.S. Provisional Patent Application No. 63/535,455, filed Aug. 30, 2023, and which is hereby incorporated by reference in its entirety.

A portion of the disclosure of this patent document contains material that is subject to copyright protection. The copyright owner has no objection to the reproduction of the patent document or the patent disclosure, as it appears in the U.S. Patent and Trademark Office (USPTO) patent file or records, but otherwise reserves all copyright rights whatsoever.

### FIELD OF THE DISCLOSURE

The present disclosure relates to a diverse modular sensor integration system with selectable sensor modules, and more particularly to apparatus, systems, and methods of implementing and operating the diverse modular sensor integration system with selectable sensor modules.

### BACKGROUND

Various approaches have been developed for integrating diverse sensors into a system. In traditional sensor integration systems, each sensor is directly connected to the system controller, resulting in a complex and inflexible architecture. This approach requires the system controller to be designed to accommodate the specific signal characteristics of each individual sensor, limiting the scalability and adaptability of the system.

Another approach involves using a single sensor module that is designed to handle multiple types of sensors. However, this approach often requires compromises in terms of signal processing capabilities and may not fully optimize the performance of each individual sensor. Additionally, the use of a single sensor module for all sensor types can result in increased complexity and cost.

Furthermore, some existing sensor integration systems utilize a fixed set of sensor modules that are permanently connected to the system controller. While this approach provides some level of flexibility, it still lacks the ability to dynamically select and configure sensor modules based on the specific requirements of the system.

However, none of these approaches have provided a comprehensive solution that combines the features described in this disclosure. The present invention addresses these limitations by providing a diverse sensor integration system that includes a system controller, at least one system sensor, and a plurality of selectable sensor modules. Each selectable sensor module is configured to receive an input signal from the system sensor, convert it into an output signal with the required signal characteristics, and transmit it to the system controller. This allows for a flexible and scalable architecture that can adapt to different sensor types and optimize the performance of each individual sensor.

Nonlimiting advantages of the present invention include the miniaturization and externalization of sensor-specific hardware and firmware to specific sensor and sensor probe locations within an expandable Modbus architecture as compared to known systems. This modularization allows for lower cost, fit-for-use application across multiple environments of application while also reducing the burden on supporting controller hardware and central processing unit (CPU) software. Further, sensor management of the present invention is improved by reducing the distance of signal transmission between a sensor analog output and the digitization of that sensor output.

### BRIEF SUMMARY

The present disclosure provides a novel diverse sensor integration system. Specifically, the present disclosure provides a novel diverse sensor integration system, as well as a method for implementing and actuating the diverse sensor integration system.

Embodiments of apparatus, methods, and systems of the present disclosure provide a solution to the shortcomings above. In particular, this disclosure provides a diverse sensor integration system. In some aspects, the techniques described herein relate to a diverse sensor integration system, including: a system controller; at least one system sensor; and a plurality of selectable sensor modules operably connected between the system controller and the at least one system sensor, each selectable sensor module configured to: receive an input signal having a set of first characteristics from the at least one system sensor; convert the input signal into an output signal having a set of second signal characteristics required by the system controller; and transmit the output signal to the system controller.

In some aspects, the techniques described herein relate to a diverse sensor integration system, wherein each selectable sensor module further includes: a sensor probe corresponding to one or more of the at least one system sensor; a control board including signal conversion circuitry; and a modular bus connector.

In some aspects, the techniques described herein relate to a diverse sensor integration system, wherein the signal conversion circuitry further includes: a sensor pigtail; an output pigtail; a microcontroller unit (MCU); a memory unit; a sensor-specific module; a resistance temperature detector (RTD) module; a communication module; and control board housing.

In some aspects, the techniques described herein relate to a diverse sensor integration system, wherein the sensor-specific module further includes: a pH module; an oxidation reduction potential (ORP) module; or an electrode conductivity (EC) module.

In some aspects, the techniques described herein relate to a diverse sensor integration system, wherein the sensor probe further includes a cable arrangement, the cable arrangement corresponds to at least one of: a dispenser arrangement; a corrosion arrangement; a toroid conductivity arrangement; an electrode conductivity arrangement; a pH arrangement; or an oxidation reduction potential (ORP) arrangement.

In some aspects, the techniques described herein relate to a diverse sensor integration system, wherein the sensor pigtail of each selectable modular sensor attachment corresponds at least to the cable arrangement.

In some aspects, the techniques described herein relate to a diverse sensor integration system, further including: a system relay operably connected to the system controller; and a dosing pump; wherein the system relay outputs a control signal for the dosing pump.

In some aspects, the techniques described herein relate to a diverse sensor integration system, wherein the system controller is a general-purpose input/output controller.

In some aspects, the techniques described herein relate to a diverse sensor integration system, wherein: the sensor probe includes a physical connector for attachment to at least one system sensor; the sensor pigtail of each selectable modular sensor attachment corresponds at least to the physical connector.

In some aspects, the techniques described herein relate to a diverse sensor integration system, wherein the output pigtail of each modular sensor attachment is identical.

In some aspects, the techniques described herein relate to a diverse sensor integration system, wherein the system is implemented on a water treatment skid.

In some aspects, the techniques described herein relate to a method of implementing a diverse sensor integration system utilizing at least one selectable modular sensor attachment, each selectable modular sensor attachment including a sensor probe, a control board, and a modular bus connector, the method including the steps of: connecting the selectable modular sensor attachment between a system sensor and a system controller; at the selectable modular sensor attachment, determining a sensor mode.

In some aspects, the techniques described herein relate to a method, wherein the step of determining the sensor mode further includes: identifying hardware of selectable modular sensor attachment through an inter-integrated circuit (I2C) communication protocol.

In some aspects, the techniques described herein relate to a method, wherein the step of identifying hardware of selectable modular sensor attachment is based at least upon a physical connector located at the sensor probe.

In some aspects, the techniques described herein relate to a method, wherein the step of identifying hardware of selectable modular sensor attachment is based at least upon a cable arrangement located at a sensor pigtail of the selectable modular sensor attachment.

In some aspects, the techniques described herein relate to a method, further including: reading a memory unit of the selectable modular sensor attachment; and wherein the step of determining the sensor mode is based at least on a sensor probe identification stored in the memory unit.

In some aspects, the techniques described herein relate to a method, further including: at the sensor probe, measuring a system value; at the control board, converting the system value to an output value; at the control board, transmitting the output value to the system controller and a system relay; and providing a control signal from the system relay to a dosing pump.

Unless otherwise defined, all technical and scientific terms used herein have the same meaning as commonly understood by one of ordinary skill in the art to which this invention belongs. The present invention may be embodied in other specific forms without departing from the spirit or essential attributes thereof, and it is therefore desired that the present embodiment be considered in all aspects as illustrative and not restrictive. Any headings utilized in the description are for convenience only and no legal or limiting effect. Numerous objects, features, and advantages of the embodiments set forth herein will be readily apparent to those skilled in the art upon reading of the following disclosure when taken in conjunction with the accompanying drawings.

### BRIEF DESCRIPTION OF THE DRAWINGS

Hereinafter, various exemplary embodiments of the disclosure are illustrated in more detail with reference to the drawings.

FIG. 1 illustrates an embodiment of the diverse sensor integration system 100 as employed in a water treatment environment, and in particular, a water treatment skid.

FIG. 2 illustrates an optional expanded embodiment of the diverse sensor integration system 100.

FIG. 3 illustrates an exemplary embodiment of the plurality of selectable sensor modules 108.

FIG. 4 illustrates an exemplary embodiment of the control board 156 in addition to the conversion circuitry 158 contained thereon for the purpose of digitizing the input analog sensor signal from the at least one system sensor 106 and converting the distinct sensor signal to an output signal that will be accepted and required by the system controller 102.

FIG. 5 illustrates an optional embodiment of one or more of the plurality of selectable sensor modules 108 as implemented as a dispenser DISP module 210.

FIG. 6 illustrates an exemplary block diagram of the control board 156, conversion circuitry 158, sensor pigtail 170, and output pigtail 172 as implemented in the determination protocol of a sensor mode.

FIG. 7 illustrates an exemplary identification protocol for identification of specific hardware of the plurality of selectable sensor modules 108 and/or the at least one system sensor 106.

### DETAILED DESCRIPTION

Reference will now be made in detail to embodiments of the present disclosure, one or more drawings of which are set forth herein. Each drawing is provided by way of explanation of the present disclosure and is not a limitation. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

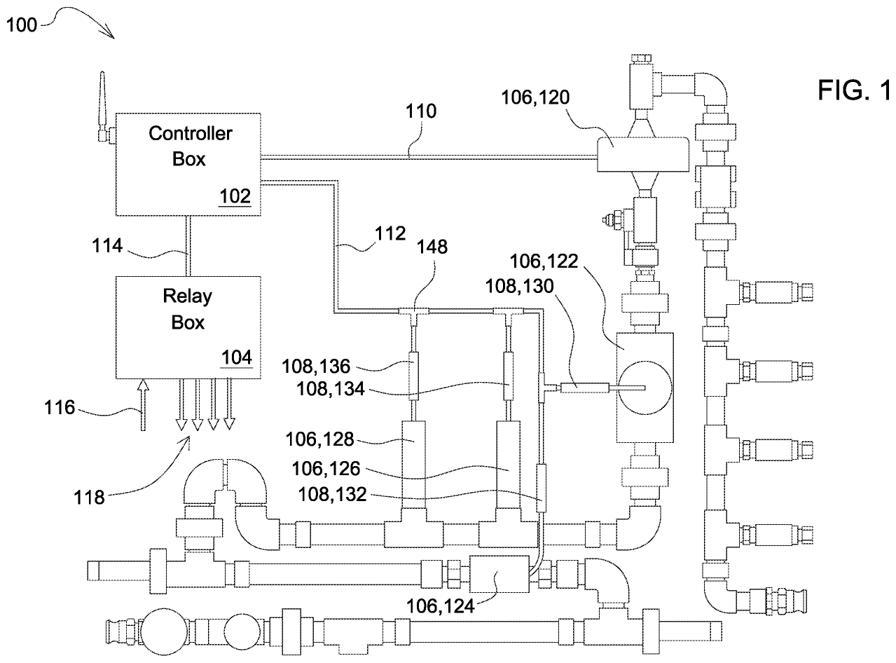

Thus, it is intended that the present disclosure covers such modifications and variations as come within the scope of the appended claims and their equivalents. Other objects, features, and aspects of the present disclosure are disclosed in, or are obvious from, the following detailed description. It is to be understood by one of ordinary skill in the art that the present discussion is a description of exemplary embodiments only and is not intended as limiting the broader aspects of the present disclosure. Referring generally to FIGS. 1-7, various exemplary embodiments may now be described of apparatuses, systems, and methods for a diverse sensor integration system 100. Various embodiments may now be described of the diverse sensor integration system 100, or one or more diverse sensor integration systems 100, and methods and systems of implementation thereof. Specifically, various embodiments may now be described of the diverse sensor integration systems 100 and a method of implementing the diverse sensor integration systems 100 in a water treatment environment 10. Where the various figures describe embodiments sharing various common elements and features with other embodiments, similar elements and features are given the same reference numerals and redundant description thereof may be omitted below.

FIG. 1 illustrates an embodiment of the diverse sensor integration system 100 as employed in a water treatment environment 10, and in particular, a water treatment skid 20. The diverse sensor integration system 100 may include a system controller 102, a system relay 104, at least one system sensor 106, and a plurality of selectable sensor modules 108. In optional embodiments, the system controller 102 may require an input signal with a distinct set of characteristics. Where the system controller 102 requires an input signal with a distinct set of characteristics, the system controller 102 may include distinctly designated input pins or may have a plurality of sensor input pins which are configured to receive either a specified or a non-specified input signal. In optional embodiments, the system controller 102 may be included as a general-purpose input/output (GPIO) controller. Where the system controller 102 is a general-purpose input/output (GPIO) controller, the signal input pins may be included as uncommitted digital signal pins, which may be used as an input or output, or both, for original equipment manufacturer (OEM) probes, meters, sensors, or any suitable sensor or control device provided within or connected to the diverse sensor integration system 100. The system controller 102 may optionally be provided with a TS-7970 central processing unit (CPU) module.

In optional embodiments, the system relay 104 may provide a 24V power supply rail line 114 to the diverse sensor integration system 100. The 24V power supply rail line 114 may be supplied by the system relay 104 to the system controller 102. In some embodiments, the 24V power supply rail line 114 may be provided through a RS-485 interface protocol such that the RS-485 protocol may supply power over bus. The RS-485 interface protocol, or Modbus RS485, may define a serial-bus-type electrical interface and physical layer for point-to-point communication of one or more devices for communication to and within the diverse sensor integration system 100. The one or more devices communicating to and within the diverse sensor integration system 100 may include RS-485 serial ports for communication purposes. The 24V power supply rail line 114 may be provided through a DC power supply such that the system relay 104 is DC-coupled to the system controller 102. In other embodiments, the 24V power supply rail line 114 may be provided through an AC power supply. The system relay 104 may further receive an AC-in 116 power supply and may further provide one or more AC-out 118 signals. The AC-in 116 and AC-out 118 couplings to and from the system relay 104 may provide communicative and/or power coupling between the system relay 104 and one or more electronic devices within the diverse sensor integration system 100 and/or to one or more electronic devices not within but connected to the diverse sensor integration system 100. For example, and as will be described further herein, the one or more AC-out 118 from the system relay 104 may be provided as relay output signals, relay output control signals in particular, that provide an input control signal to one or more dosing pumps 144 within or connected to the diverse sensor integration system 100 to control the introduction and dosing of chemicals or agents into the diverse sensor integration system 100.

The at least one system sensor 106 may include a number of different sensors disposed throughout and/or connected to the diverse sensor integration system 100. One, some, or all of the at least one system sensor 106 may be original equipment manufacturer (OEM) sensors. For example, the at least one system sensor 106 may include a fluorometer sensor 120 to measure the concentration of fluorescent dyes, specifically PTSA or Fluorescein, in a water sample within the water treatment environment 10.

In optional embodiments, the fluorometer sensor 120 may be an original equipment manufacturer (OEM) fluorometer, including for example products advertised under the Oxamine® water treatment systems.

The at least one system sensor 106 may also include a power sensor and/or electrode conductivity EC sensor 122 to measure the conductivity of the water and/or the current, voltage, or both, of the diverse sensor integration system 100 and may for example include a toroidal conductivity (T-COND) sensor module.

The at least one system sensor 106 may further include a flow meter sensor 124 to measure the volume and/or rate of fluid moving through the water treatment environment 10.

The at least one system sensor 106 may also include an oxidation reduction potential ORP sensor 126, or redox sensor, to measure the relative oxidation and reduction properties of water within the water treatment environment 10.

The at least one system sensor 106 may further include a pH sensor 128 to measure the pH, or relative acidity of water within the water treatment environment 10.

As will be appreciated by one of ordinary skill in the art, the diverse sensor integration system 100 may include additional sensors included within the at least one system sensor 106 to measure relevant properties and characteristics of water or other liquid being within or connected to the water treatment environment 10.

The diverse sensor integration system 100 may include plurality of selectable sensor modules 108 corresponding to and operably connected to the at least one system sensor 106. The plurality of selectable sensor modules 108 may be provided as or may include Atlas Sensor Technologies sensor modules as hardware to be implement within the plurality of selectable sensor modules 108. By way of a nonlimiting example, the electrode conductivity EC sensor 122 may be operably connected to an electrode conductivity EC sensor module 130. The electrode conductivity EC sensor module 130 may be provided as a T-COND sensor module or an Atlas EC sensor module. The flow meter sensor 124 may be operably connected to a flow meter sensor module 132. The oxidation reduction potential ORP sensor 126 may be operably connected to an oxidation reduction potential ORP sensor module 134. The oxidation reduction potential ORP sensor module 134 may be provided as an Atlas ORP sensor module. The pH sensor 128 may be operably connected to a pH sensor module 136. The pH sensor module 136 may be provided as an Atlas pH sensor module.

The plurality of selectable sensor modules 108 may be operably connected to the system controller 102 by a number of communication signal and power lines. As an example, the fluorometer sensor 120 may be connected to the system controller 102 via a 7V rail line 110. Other sensors, including the electroconductivity EC sensor 122, flow meter sensor 124, oxidation reduction potential ORP sensor 126, and pH sensor 128, may be connected to the system controller 102 via a 12V rail line 112. The communication signal and power lines, including the 7V rail line 110 and 12V rail line 112, may include a RS-485 protocol for communication. Additionally, the plurality of selectable sensor modules 108 may be connected from the at least one system sensor 106 to the system controller 102 by one or more M12 tee connectors 148. The tee connectors 148 may optionally be any suitable connector to provide operable and communicative coupling between the plurality of selectable sensor modules 108 and the system controller 102.

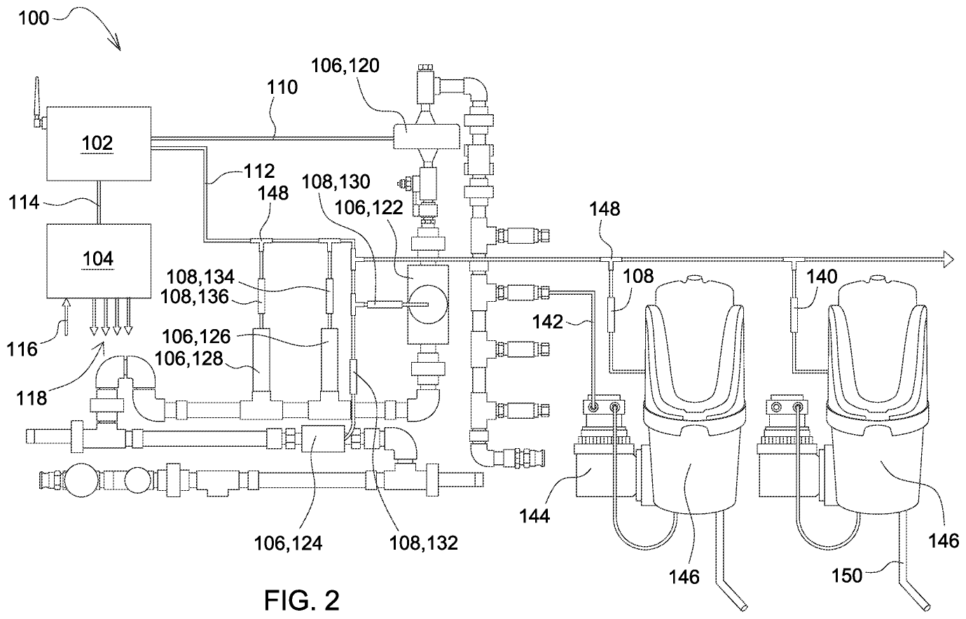

FIG. 2 illustrates an optional expanded embodiment of the diverse sensor integration system 100. As illustrated in FIG. 2, the diverse sensor integration system 100 may be provided as an expandable Modbus architecture that may include additional sensors, controllers, and various electronic and physical devices to provide measuring and treatment within or connected to the water treatment environment 10. The diverse sensor integration system 100 may further include a polyvinyl chloride PVC level sensor (not pictured) and a polyvinyl chloride PVC level sensor module 140 operably connected to the polyvinyl chloride PVC level sensor.

The diverse sensor integration system 100 may additionally include one or more chemical feeds 142 to provide one or more chemicals, treating agents, or other elements to alter the composition, properties, and/or characteristics of water within the water treatment environment 10. The chemical feed 142 may provide a chemical input to one or more dosing pump 144. The one or more dosing pumps 144 may provide a controlled dosage of chemicals to the diverse sensor integration system 100 via one or more dispensers 146. The diverse sensor integration system 100 may also include one or more overflow drains 150 to provide a safety outlet for chemicals from the one or more chemical feeds 142 from being introduced into the water treatment environment 10 in concentrations of excess. In optional embodiments, the one or more overflow drains 150 may be provided at the one or more dispensers 146 such that excess one or more chemical feeds 142 from the one or more dosing pumps 144 may not be introduced into the water treatment environment 10.

In optional embodiments, the plurality of selectable sensor modules 108 may include various smart cables, including one or more dispenser DISP modules 210, corrosion modules (not pictured), and toroid cables (not pictured). The one or more dispenser DISP modules 210 and the toroid cables (not pictured) may be operably and communicatively coupled to the remainder of the diverse sensor integration system 100 through 7V rail lines 110, 12V rail lines 112, and/or 24V rail lines 114. Dispenser DISP modules 210 may be operably coupled with the one or more dispensers 146. The corrosion module (not pictured) may be operably connected to a linear polarization resistance LPR sensor (not shown), which is configured to monitor corrosion within the water treatment environment 10. In further embodiments, the plurality of selectable sensor modules 108 may include a toroid cable for general purpose operative and communicative coupling within, to, and from the diverse sensor integration system 100.

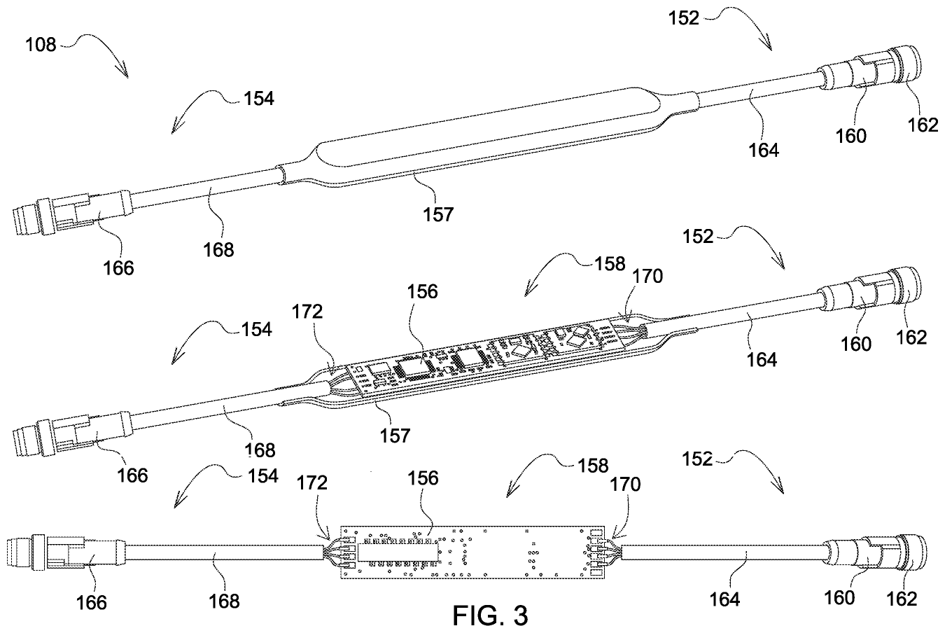

FIG. 3 illustrates an exemplary embodiment of the plurality of selectable sensor modules 108. As illustratively shown in FIG. 3, the plurality of selectable sensor modules 108 may include a sensor portion 152, an output portion 154 and a control board 156. The control board 156 may be provided within a control board housing 157. The control board housing 157 may be formed as a two-part 3D printed clamshell housing and may be formed of any suitable material that protects the control board 156 from environmental conditions present within the water treatment environment 10 as known to those of ordinary skill in the art. The control board 156 may also include conversion circuitry 158 for converting and digitizing an analog signal from the at least one system sensor 106 to the system controller 102 and will be described further herein.

The sensor portion 152 may include a sensor probe 160 with a sensor connector 162 and may further include a sensor end cable 164. The sensor connector 162 may correspond to one or more of the at least one system sensor 106. In optional embodiments, the sensor connector 162 may distinctly correspond to the at least one system sensor 106 such that one of the plurality of selectable sensor modules 108 will be selected for a particular one of the at least one system sensor 106 based at least upon the physical configuration of the sensor connector 162. As an illustrative example, the sensor connector 162 for the oxidation reduction potential ORP sensor module 134 may correspond to the oxidation reduction potential ORP sensor 126 and the oxidation reduction potential ORP sensor 126 only. In optional embodiments, the sensor probe 160 and sensor connector 162 may be formed as an M12 circular cable pigtail and connector for operable and communicative coupling to the at least one system sensor 106.

The output portion 154 may include a system controller connector 166, which may optionally be provided as a modular bus connector, and an output end cable 168. The system controller connector 166 may similarly be formed as an M12 circular cable pigtail and connector for operable and communicative coupling to the various rail lines 110, 112, 114 that provide power, operative, and communicative coupling within the diverse sensor integration system 100. Together, the plurality of selectable sensor modules 108 with the output portion 154 connected to various rail lines 110, 112, 114 through a number of tee connectors 148, all of which will operatively couple to the system controller 102, for a Modbus daisy chain.

The sensor portion 152 may additionally include a sensor pigtail 170 to connect the sensor probe 160, sensor connector 162, and sensor end cable 164 to the control board 156. Similarly, the output portion 154 may additionally include an output pigtail 172 to connect the control board 156 to the system controller connector 166 through the output end cable 168.

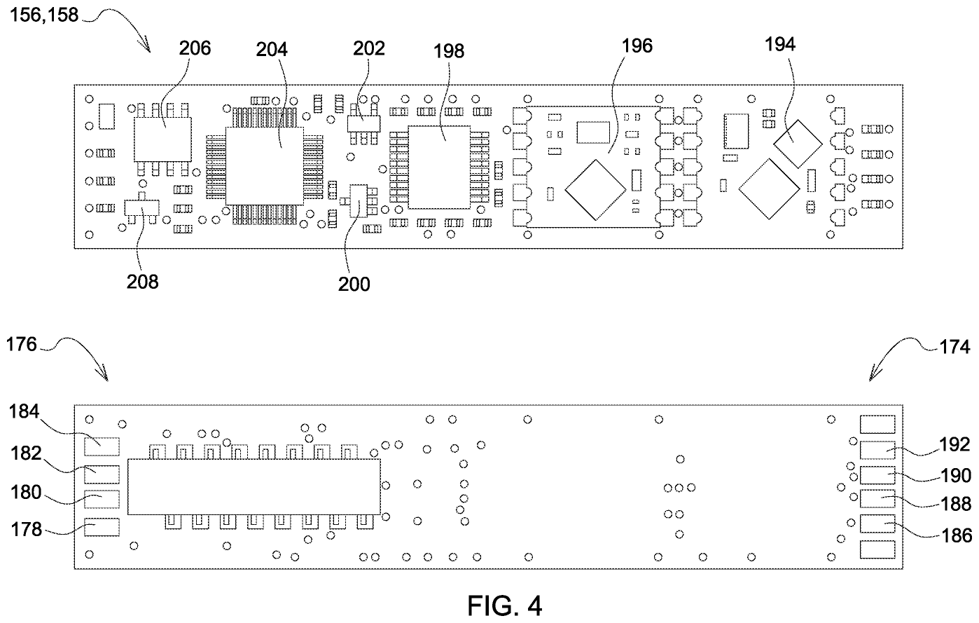

FIG. 4 illustrates an exemplary embodiment of the control board 156 in addition to the conversion circuitry 158 contained thereon for the purpose of digitizing the input analog sensor signal from the at least one system sensor 106 and converting the distinct sensor signal to an output signal that will be accepted and required by the system controller 102. The control board 156 may include a control board sensor connector 174 provided as a plurality of connection pads to allow connection to the sensor pigtail 170 and may also include a control board output connector 176 similarly provided as a plurality of connection pads to allow connection to the output pigtail 172.

The conversion circuitry 158 may include a number of components, some of which may be specific to the at least one system sensor 106 to which the plurality of selectable sensor modules 108 is adapted to be connected. The conversion circuitry 158 may include a sensor specific module 194. In the diverse sensor integration system 100 the plurality of selectable sensor modules 108 may include at least three modules, including the oxidation reduction potential ORP sensor 126, the pH sensor 128, and the electrode conductivity EC sensor module 130. Accordingly, the control board 156 and conversion circuitry 158 for each will include a sensor specific module 194 that corresponds to an ORP module, a pH module, and an EC module, respectively. The sensor specific module 194 may be distinctly included within one or more of the plurality of selectable sensor modules 108 to provide specific sensor compatibility. Accordingly, other of the sensor specific module 194 will be included based upon additional of the at least one system sensor 106 included within the diverse sensor integration system 100. The conversion circuitry 158 may also include an RTD module 196. The RTD module 196 may be populated or may not be populated based upon a specific one of the plurality of selectable sensor modules 108. As an illustrative example, the electrode conductivity EC sensor module 130 may include a RTD module 196 that is populated. The oxidation reduction potential ORP sensor module 134 and the pH sensor module 136 may include the RTD module 196 that is not populated.

The conversion circuitry 158 may further include a power isolation module 198. The conversion circuitry 158 may also include a 5V regulator 200. The conversion circuitry 158 may further include a memory unit 202 that may store information provided to accomplish implementation of the plurality of selectable sensor modules 108, including identification of the hardware within the sensor probe 160. The memory unit 202 may be provided as RAM memory, flash memory, ROM memory, EPROM memory, or EEPROM memory. The conversion circuitry 158 may also include a microcontroller unit MCU 204 which may provide for processing of the input sensor signal and that may provide for identification of hardware of the at least one system sensor 106 and the plurality of selectable sensor modules 108 upon power as described further herein. The conversion circuitry 158 may further include a transceiver 206 to transmit the converted and digitized analog sensor signal as an output signal acceptable by and required by the system controller 102. The conversion circuitry 158 may also include a 3.3V regulator 208. Both the 5V regulator 200 and the 3.3V regulator may provide for regulation of voltage during power fluctuations and variations in loads as they exist both in AC as well as DC voltages.

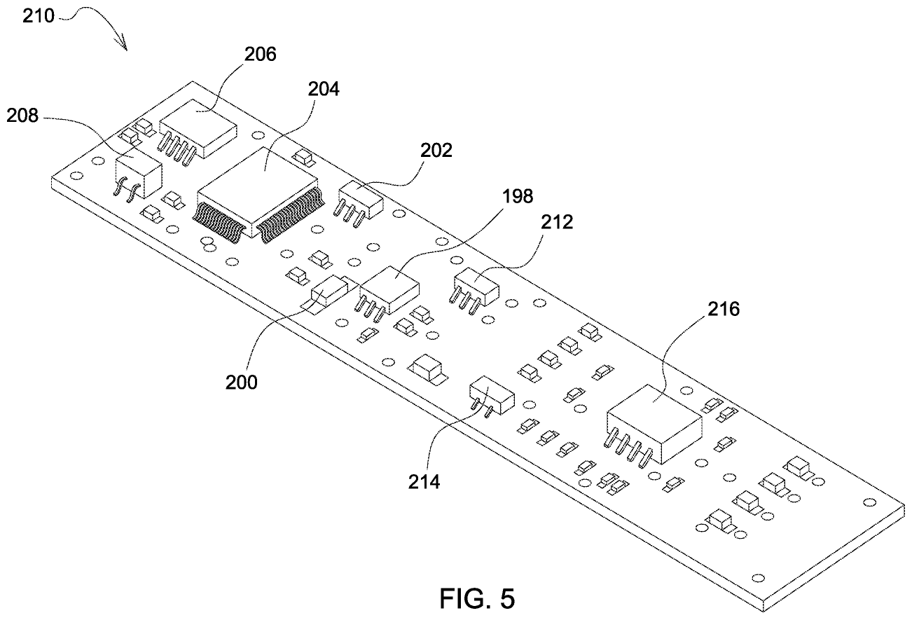

FIG. 5 illustrates an optional embodiment of one or more of the plurality of selectable sensor modules 108 as implemented as a dispenser DISP module 210. As introduced above, the one or more dispenser DISP modules 210 are similar to the rest of the plurality of selectable sensor modules 108. The one or more dispenser DISP modules 210 tie into the existing modbus daisy chain, to any one or more of the 7V, 12V, or the 24V rail lines 110, 112, 114. The one or more dispenser DISP modules 210 may also be compatible with various electronic devices employed within the diverse sensor integration system 100, including Aquaman controllers and legacy modbus registers. The one or more dispenser DISP modules 210 may also include different or unique of the sensor connector 162 and/or the system controller connector 166 for purposes of identification of the one or more dispenser DISP modules 210. Like the control board 156 and the conversion circuitry 158, the one or more dispenser DISP modules 210 may include a temperature sensor 198, a 5V regulator 214, a memory unit 202, a microcontroller unit MCU 204, a 3.3V regulator 208, and additional components as may be necessary to provide operative and communicative coupling between the one or more dispensers 146 and the system controller 102.

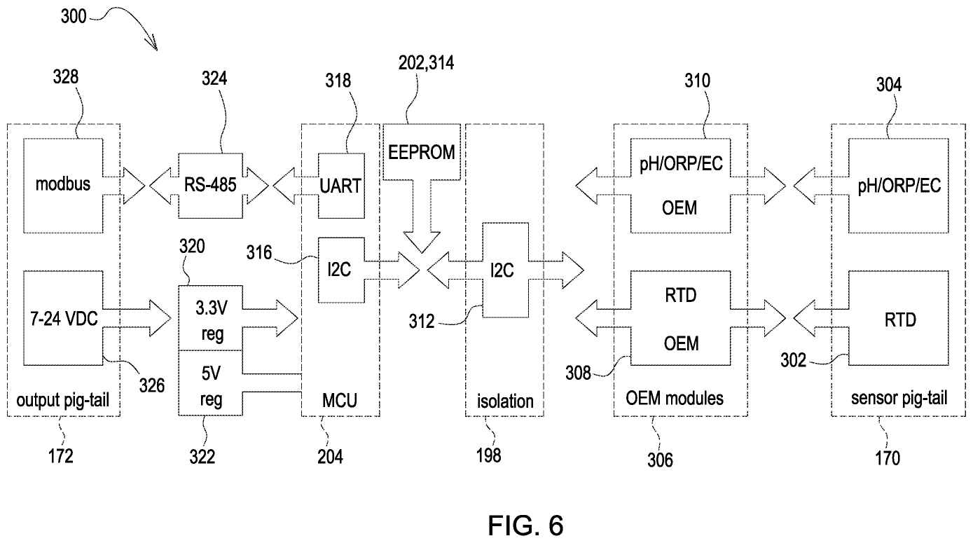

FIG. 6 illustrates an exemplary block diagram of the control board 156, conversion circuitry 158, sensor pigtail 170, and output pigtail 172 as implemented in the determination protocol of a sensor mode. A sensor mode determination protocol 300 includes in the sensor pigtail 170 an RTD identifier 302 to identify an incoming RTD signal and a sensor identifier 304 to identify an incoming analog sensor signal, which may illustratively correspond to the electroconductivity EC sensor 122, the oxidation reduction potential ORP sensor 126, and/or the pH sensor 128. As introduced above, the electrode conductivity EC sensor 122 may provide an incoming signal indicative of electrode conductivity and an RTD identifier 302 with a populated RTD signal. The oxidation reduction potential ORP sensor 126 may provide an incoming signal indicative of oxidation reduction potential ORP and an RTD identifier 302 with a nonpopulated RTD signal. The pH sensor 128 may provide an incoming signal indicative of pH and an RTD identifier 302 with a nonpopulated RTD signal.

An OEM module 306, which may be the sensor specific module 194, includes an OEM RTD module 308 and an OEM specific sensor module 310. The OEM RTD module 308 may receive and transmit information to and from the RTD identifier 302 and provide a populated or unpopulated RTD signal. The OEM specific sensor module 310 may receive and transmit information to and from the sensor identifier 304 and process the information from the sensor identifier 304 to determine the hardware within the selected one of the plurality of selectable sensor modules 108 as it correspond to the specific sensor of the at least one system sensor 106.

The power isolation module 198 may include a power isolation I2C protocol module 312 to transmit and receive information to and from the OEM module 306.

The memory unit 202 may be included and provide a memory signal 314. The power isolation I2C protocol module 312 may communicate with the memory unit 202 and memory signal 314 in identifying the sensor mode. For example, the memory unit 202 may store a predetermined memory signal 314 that is indicative of the specific hardware employed within the particular one of the plurality of selectable sensor modules 108 and provide identification thereof within the sensor mode determination protocol 300 upon power.

The microcontroller unit MCU 204 may include an MCU I2C protocol module 316 and a UART protocol module 318. The MCU I2C protocol module 316 may communicate with the memory unit 202, particularly the memory signal 314, and the power isolation I2C protocol module 312. The microcontroller unit MCU 204 may additionally provide a signal to the power isolation module 198 indicative of a power or voltage regulation as described herein.

The sensor mode determination protocol 300 may include a 3.3V regulation 320 and a 5V regulation 322 for regulation of power or voltage. Both the 3.3V regulation 320 and the 5V regulation 322 may provide a power regulation signal to the microcontroller unit MCU 204. The microcontroller unit MCU 204 may further provide the power regulation signal to the power isolation module 198 based upon at least the 3.3V regulation 320 and the 5V regulation 322.

The sensor mode determination protocol 300 further includes an RS-485 protocol module 324. The RS-485 protocol module 324 may communicate with the UART protocol module 318 of the microcontroller unit MCU 204.

The sensor mode determination protocol 300 also includes the output pigtail 172, which includes a 7-24 VDC input module 326 and a modbus module 328. The 7-24 VDC module 326 may provide power to the 3.3V regulation 320, the 5V regulation 322, or both. The modbus module 328 may communicate with the RS-485 protocol module 324.

In operation, the sensor mode determination protocol 300 may operate to determine a sensor mode through an I2C communication protocol. The sensor mode determination protocol 300 may operate to determine a sensor mode by identifying specific hardware of the one of the plurality of selectable sensor modules 108 based at least upon sensor connector 162, or physical connector, at the sensor probe 160. The sensor mode determination protocol 300 may operate to determine a sensor mode by identifying specific hardware of the one of plurality of selectable sensor modules 108 based at least upon a specific cable arrangement within the sensor end cable 164 located at the sensor pigtail 170.

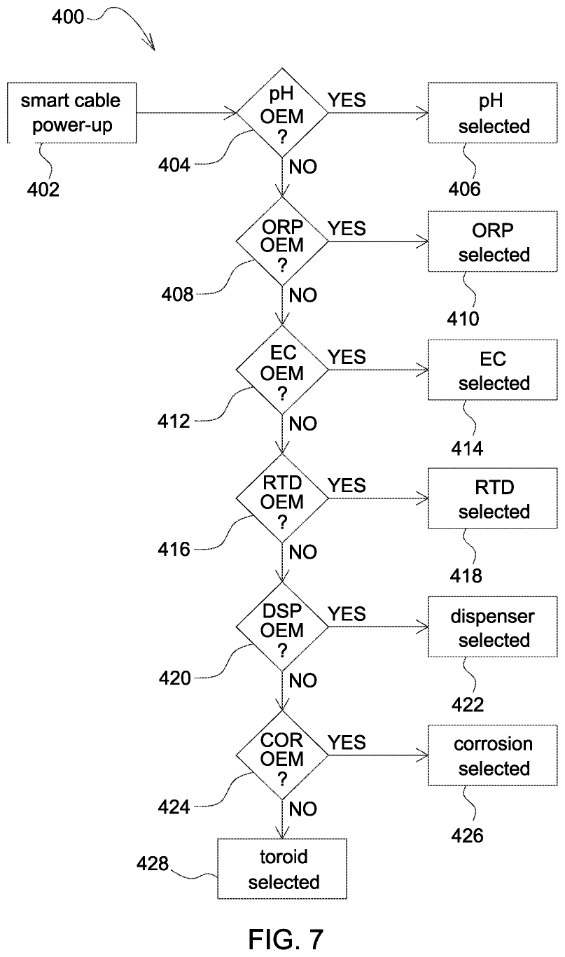

FIG. 7 illustrates an exemplary identification protocol for identification of specific hardware of the plurality of selectable sensor modules 108 and/or the at least one system sensor 106. An identification protocol 400 may begin with a power-up 402 of one of the plurality of selectable sensor modules 108. After power-up 402, the identification protocol 400 may continue with a pH OEM inquiry 404 to determine if OEM pH hardware is employed within the at least one system sensor 106 and/or the one of the plurality of selectable sensor modules 108. If the pH OEM inquiry 404 results in a YES verification, the step of a pH sensor mode selection 406 is completed. If the pH OEM inquiry 404 results in a NO verification, an ORP OEM inquiry 408 is performed to determine if OEM ORP hardware is employed within the at least one system sensor 106 and/or the one of the plurality of selectable sensor modules 108. If the ORP OEM inquiry 408 results in a YES verification, the step of an ORP sensor mode selection 410 is completed. If the ORP OEM inquiry 408 results in a NO verification, an EC OEM inquiry 412 is performed to determine if OEM EC hardware is employed within the at least one system sensor 106 and/or the one of the plurality of selectable sensor modules 108. If the EC OEM inquiry 412 results in a YES verification, the step of an EC sensor mode selection 414 is completed. If the EC OEM inquiry 412 results in a NO verification, an RTD OEM inquiry 416 is performed to determine if OEM RTD hardware is employed within the at least one system sensor 106 and/or the one of the plurality of selectable sensor modules 108. If the RTD OEM inquiry 416 results in a YES verification, the step of an RTD module population 418 is completed. If the RTD OEM inquiry 416 results in a NO verification, a dispenser DSP ADC inquiry 420 is performed to determine with a dispenser is being employed within the at least one system sensor 106 and/or the one of the plurality of selectable sensor modules 108. If the dispenser DSP ADC inquiry 420 results in a YES verification, the step of a dispenser mode selection 422 is completed. If the dispenser DSP ADC inquiry 420 results in a NO verification, a corrosion COR ADC inquiry 424 is performed to determine whether corrosion hardware is employed within the at least one system sensor 106 and/or the one of the plurality of selectable sensor modules 108. If the corrosion COR ADC inquiry 424 results in a NO verification, the step of a toroid mode selection 428 is completed.

The term “controller” as used herein may refer to at least general-purpose or specific-purpose processing devices, such as a central processing unit, and/or logic as may be understood by one of skill in the art, including but not limited to a microprocessor, a microcontroller, a state machine, and the like. The processor can also be implemented as a combination of computing devices, e.g., a combination of a digital signal processor (DSP) and a microprocessor, a plurality of microprocessors, one or more microprocessors in conjunction with a DSP core, or any other such configuration.

Terms such as “a,” “an,” and “the” are not intended to refer to only a singular entity, but rather include the general class of which a specific example may be used for illustration.

The phrases “in one embodiment,” “in optional embodiment(s),” and “in an exemplary embodiment,” or variations thereof, as used herein does not necessarily refer to the same embodiment, although it may.

As used herein, the phrases “one or more,” “at least one,” “at least one of,” and “one or more of,” or variations thereof, when used with a list of items, means that different combinations of one or more of the items may be used and only one of each item in the list may be needed. For example, “one or more of” item A, item B, and item C may include, for example, without limitation, item A or item A and item B. This example also may include item A, item B, and item C, or item B and item C.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements, and/or states. The conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. Thus, such conditional language is not generally intended to imply that features, elements, and/or states are in any way required for one or more embodiments, whether these features, elements, and/or states are included or are to be performed in any particular embodiment.

The previous detailed description has been provided for the purposes of illustration and description. Thus, although there have been described particular embodiments of a new and useful invention, it is not intended that such references be construed as limitations upon the scope of this disclosure except as set forth in the following claims. Thus, it is seen that the apparatus of the present disclosure readily achieves the ends and advantages mentioned as well as those inherent therein. While certain preferred embodiments of the disclosure have been illustrated and described for present purposes, numerous changes in the arrangement and construction of parts and steps may be made by those skilled in the art, which changes are encompassed within the scope and spirit of the present disclosure as defined by the appended claims.

What is claimed is:

1. A diverse sensor integration system for collecting data from one or more sensors at respective data collection nodes in an industrial production site, wherein each of the one or more sensors produces signals according to a respective signal protocol, comprising:
a system controller; and
a plurality of selectable sensor modules, wherein each sensor module is configured, upon being operably connected between the system controller and a respective one of the at least one system sensor, to:
receive an input signal having a set of first characteristics corresponding at least in part to the respective signal protocol from the at least one system sensor;
convert the input signal into an output signal having a set of second signal characteristics corresponding at least in part to a signal protocol associated with the system controller; and
transmit the output signal to the system controller,
wherein the system controller is configured to perform operations based on the output signals from the one or more sensor modules connected thereto, independent of the respective signal protocol for each of the one or more sensors.

2. The diverse sensor integration system of claim 1, wherein each selectable sensor module further comprises:
a sensor probe corresponding to one or more of the at least one system sensor;
a control board comprising a signal conversion circuitry; and
a system controller connector.

3. The diverse sensor integration system of claim 2, wherein the signal conversion circuitry further comprises:
a sensor pigtail;
an output pigtail;
a microcontroller unit (MCU);
a memory unit;
a sensor-specific module;
a resistance temperature detector (RTD) module;
a communication module; and
control board housing.

4. The diverse sensor integration system of claim 3, wherein the sensor-specific module further comprises:
a pH module;
an oxidation reduction potential (ORP) module; or
an electrode conductivity (EC) module.

5. The diverse sensor integration system of claim 2, wherein the sensor probe further comprises a cable arrangement, the cable arrangement corresponds to at least one of:
a dispenser arrangement;
a corrosion arrangement;
a toroid conductivity arrangement;
an electrode conductivity arrangement;
a pH arrangement; or
an oxidation reduction potential (ORP) arrangement.

6. The diverse sensor integration system of claim 3, wherein:
the sensor pigtail of each selectable modular sensor attachment corresponds at least to the cable arrangement.

7. The diverse sensor integration system of claim 1, further comprising:
a system relay operably connected to the system controller; and
a dosing pump;
wherein the system relay outputs a control signal for the dosing pump.

8. The diverse sensor integration system of claim 1, wherein:
the system controller is a general-purpose input/output controller.

9. The diverse sensor integration system of claim 3, wherein:
the sensor probe comprises a physical connector for attachment at the at least one system sensor; and
the sensor pigtail of each selectable modular sensor attachment corresponds at least to the physical connector.

10. The diverse sensor integration system of claim 3, wherein:
the output pigtail of each modular sensor attachment is identical.

11. The diverse sensor integration system of claim 1, wherein:
the system is implemented on a water treatment skid.

12. A method of implementing a diverse sensor integration system utilizing at least one selectable modular sensor attachment, each selectable modular sensor attachment comprising a sensor probe, a control board, and a system controller connector, the method comprising the steps of:
connecting the selectable modular sensor attachment between a system sensor and a system controller; and
at the selectable modular sensor attachment, determining a sensor mode.

13. The method of claim 12, wherein the step of determining the sensor mode further comprises:
identifying hardware of selectable modular sensor attachment through an I2C communication protocol.

14. The method of claim 12, wherein:
the step of identifying hardware of selectable modular sensor attachment is based at least upon a physical connector located at the sensor probe.

15. The method of claim 12, wherein:
the step of identifying hardware of selectable modular sensor attachment is based at least upon a cable arrangement located at a sensor pigtail of the selectable modular sensor attachment.

16. The method of claim 12, further comprising:
reading a memory unit of the selectable modular sensor attachment; and
wherein the step of determining the sensor mode is based at least on a sensor probe identification stored in the memory unit.

17. The method of claim 12, further comprising:
at the sensor probe, measuring a system value;
at the control board, converting the system value to an output value;
at the control board, transmitting the output value to the system controller and a system relay; and
providing a control signal from the system relay to a dosing pump.
